(19) United States
(12) Patent Application Publication
(10) Pub. No.: US 2024/0148623 A1
(43) Pub. Date: May 9, 2024

(54) GADUSOL AND GADUSPORINE COMPOUND FORMULATIONS FOR TOPICALS

(71) Applicant: GADUSOL LABORATORIES, INC., Corvallis, OR (US)

(72) Inventors: Alan T. Bakalinsky, Corvallis, OR (US); Khaled H. Almabruk, Corvallis, OR (US); Kathryn Pettinger, Corvallis, OR (US); Irwin Palefsky, Elwood, NY (US); Atchara Sripeng, Corvallis, OR (US); Nathan Alexander, Corvallis, OR (US)

(21) Appl. No.: 18/548,700

(22) PCT Filed: Mar. 1, 2022
(86) PCT No.: PCT/US2022/018377

§ 371 Date: Sep. 1, 2023

Related U.S. Application Data
(60) Provisional application No. 63/155,104, filed on Mar. 1, 2021.

(51) Int. Cl. A61K 8/27; A61K 8/06; A61K 8/35; A61Q 17/04

(52) U.S. Cl. CPC A61K 8/27; A61K 8/062; A61K 8/35; A61Q 17/04

(57) ABSTRACT

Compound formulations include gadusol or gadusporine that can be used as topicals, such as sunscreens or filters that protect human and animal skin against harmful light like UVA, and UVB, cosmetics, and skin care products. In some examples, gadusol or gadusporine are ≤0.5% w/w, 0.5-2.0% w/w of the compound formulation. Other compound formulations include gadusol or gadusporine at >2.0% w/w of the compound formulation.

### CROSS-REFERENCE TO RELATED APPLICATIONS

This application is a continuation of co-pending U.S. Patent Application Ser. No. 63/155,104, filed Mar. 1, 2021, the contents of which are herein incorporated by reference in their entirety.

### BACKGROUND

Sunburn, aging and skin cancers result from overexposure of human skin to ultraviolet (UV) radiation. Broad spectrum sunscreens protect from UVB radiation (280 to 320 nm), which causes skin cancer and sunburn, as well as UVA radiation (320 to 400 nm), which contributes to skin cancer and causes aging and wrinkling of skin.

The FDA's list of market-approved compounds includes two UVA filters: avobenzone and zinc oxide and six compounds that absorb or block UVB radiation, including oxybenzone, octinoxate, octisalate, homosalate, octocrylene, titanium dioxide, and zinc oxide. Current sunscreen products typically include at least one reflective particle (titanium oxide or zinc oxide) and one or more aromatic organic compounds in an oil/water emulsion.

Organisms, particularly microorganisms, algae and many marine invertebrates, have developed their own natural sunscreens. Many compounds in the class of mycosporine-like aminoacids (MAAs) absorb UVA without producing free radicals and singlet oxygen. For example, porphyra-334 and shinorine are produced by the red algae Porphyra umbilicalis (sold as “nori” seaweed) and are known as natural sunscreens. There is increasing interest in these natural and organic “botanicals” for skin use.

Synthetic UVA/UVB filters may include oxybenzones, octinoxates, benzophenones, methoxycinnamates, phenylbenzimidazoles, phthalylidene dicamphor sulfonic acid, and biphenyltriazines. Many of these synthetic compounds and their metabolites may be harmful to humans, e.g., are intercalating agents and can be mutagens, are anti-estrogens, and may alter metabolism of other drugs. The synthetic compounds are also more generally toxic in the environment to a wide range of organisms. Sunscreens compounded with inorganic particles, e.g., TiO₂, ZnO, can have a viscous skin feel and white appearance and as such, are often less desirable to consumers.

The art could benefit from a new sun filter or sunscreen that is safe and effective.

### BRIEF DESCRIPTION OF THE DRAWINGS

FIGS. 1A-1C show the chemical structures for gadusporines A, B, and C, respectively.

FIG. 2 shows an example UV spectrum for gadusol and gadusporine A.

FIG. 3 shows an example UV spectrum for gadusol and gadusporine A across UV-A through UV-C.

FIG. 4 shows a block diagram that illustrates components of a sample topical compound formulation that includes gadusol or gadusporine.

### DETAILED DESCRIPTION

Described herein are systems and methods for developing topical formulas using gadusol and gadusporine. Gadusol and gadusporines are UVB- and UVA-protective compounds, respectively. They provide antioxidant functionality for the skin or associated tissues, because of their free radical-scavenging activity. Furthermore, inclusion of one or more of these sun blocking or screening agents in a composition, such as a topical sunscreen or cosmetic allows for a more transparent, all natural sunscreen compared with metal oxide-containing formulations. In formulations, gadusol contributes to ease of spreadability and transparency because it is soluble and transparent in the water phase. The combination of gadusol with ZnO in such formulations will allow use of reduced amounts of insoluble ZnO particles that will result in increased transparency, uniform spreadability, and less whiteness, but with the same SPF activity. These negative sensory characteristics of ZnO-containing sunscreen formulations are considered to be problematic by the cosmetics industry because consumers prefer easy-to-spread, transparent formulations.

The yeast Saccharomyces cerevisiae may be used to produce gadusol via 2-epi-5-epi-valiolone synthase (EEVS) and methyltransferase/oxidoreductase (MT-Ox) as described in Osborn et al., “De novo synthesis of a sunscreen compound in vertebrates,” elife. 2015; 4:e05919, incorporated by reference herein. The genes encoding these enzymes were introduced into yeast via integration into a chromosome, where they were expressed to produce EEVS and MT-Ox, allowing the yeast to use SHIP to form gadusol. This yeast strain was initially grown under anaerobic conditions with glucose as a carbon source for production of gadusol. This resulted in yields of about 50 mg/L, which may be improved using different growth conditions. Gadusol along with mycosporine-like amino acids (MAAs) analogs (Gadusporine A, Gadusporine B, and Gadusporine C, illustrated below) may be produced using bacteria (Streptomyces coelicolor). For example, MAA analogs as well as gadusol were produced using an expression vector in a bacterial host as described in Osborn et al., “Interkingdom Genetic Mix-and-Match To Produce Novel Sunscreens,” ACS Synth. Biol. 2019, 8, 11, 2464-2471, Nov. 5, 2019, incorporated by reference herein. Generally the yields from the bacterial host are greater than that of yeast cultures. Methods of purification of the resultant end products (MAAs and gadusol) are also documented elsewhere.

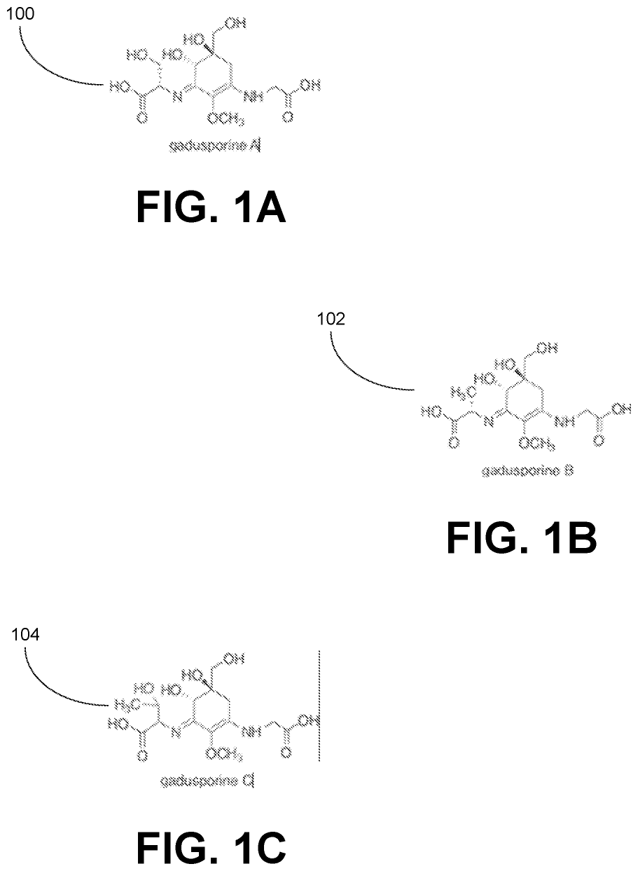

The chemical structures of gadusporines A 100, B 102, and C 104 isolated from an example bacterial production process are illustrated in FIGS. 1A-1A.

Gadusol, gadusporine A 100, gadusporine, B 102, and gadusporine C 104 may be isolated by column purification of Streptomyces coelicolor culture broth (as described in the above publication). Gadusporines A 100 and B 102 both had a UV maximum at 338 nm under acidic conditions.

As shown above, gadusporine A 100 includes L-serine and glycine attached to the gadusol core.

Despite the only difference being a hydroxy group, there are a few notable differences between shinorine and gadusporine A 100. Shinorine has been reported to have an absorption max=330 nm at pH 1.7 and an absorption max=334 nm at pH 7. Gadusporine A 100 has an absorption max=338 at pH 2.0 and an absorption max=340 nm at pH 7.0, thus exhibiting a narrower range of UV absorbance across pH values. Gadusporine A 100 has a molar extinction coefficient (□□=49,400, as measured in 50 mM phosphate buffer at pH 7.0, which is slightly higher than that of shinorine (□□=44,000), and thus has stronger UV absorbance activity.

Gadusporine B 102 contains L-alanine and glycine, making gadusporine B 102 an analog of mycosporine-glycine-alanine. Like gadusporine A 100, gadusporine B 102 also exists in solution as a mixture of two isomers (3:1 ratio) and is not very soluble in methanol.

Gadusporine C 104 was also separated from gadusol and gadusporine B 102. However, only minute amounts (less than 0.1 mg) were obtained which precluded full structural elucidation, including NMR spectra. Gadusporine C's 104 predicted formula is a hydroxylated porphyra-334 analog. The structure for gadusporine C 104 is predicted based on the elucidated structures for gadusporines A 100 and B 102.

Sunscreens and Related Uses

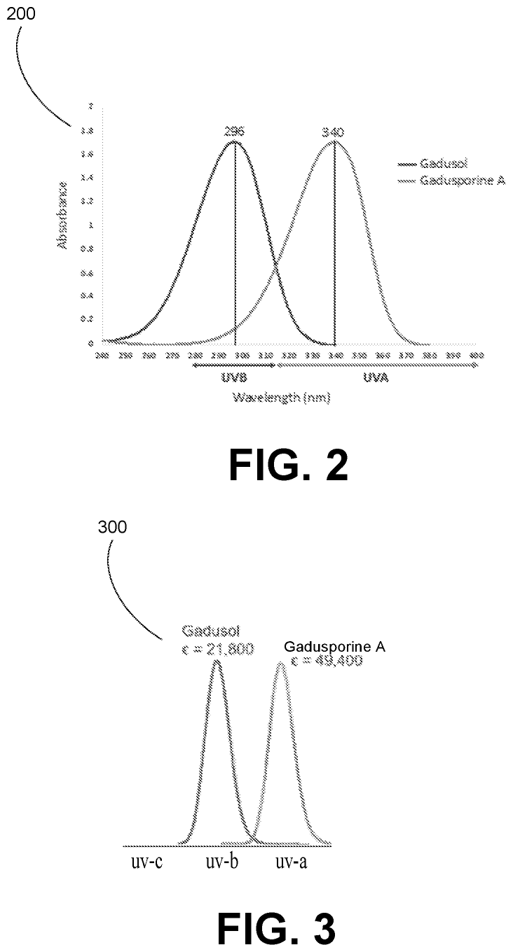

Gadusol and gadusporines A 100, B 102, and C 104 absorb UV radiation at different wavelengths, allowing them to be used in combination to form broad spectrum sunscreens. An example of UV spectrum coverage 200 for gadusol and gadusporine A 100 is illustrated in FIG. 2.

Gadusol alone may be useful in sunscreen formulations. Therefore, the ability to produce and purify gadusol in sufficient quantity are prerequisites for inclusion in such compositions. Gadusol can be both an SPF booster or a new sunscreen.

Boosters are defined in the industry as ingredients that increase the overall SPF value of a sunscreen formulation, but alone contribute <2 SPF units in the absence of the active sunscreen ingredient. Gadusol and gadusporine both absorb UVB and UVA directly, respectively. In some example compound formulations, a small amount of gadusol is added such that it provides <2 SPF units as a standalone component. For this reason, in this formulation, gadusol serves as a booster. Other example boosters increase the solubility or stability of the active sunscreen or increase its ability to scatter UV. When this small amount of gadusol is added to a formulation with 10% zinc oxide for example, the SPF value of the formulation doubles. This formulation allows for lower levels of zinc oxide to be used, which improves the application of the formulation on the skin because too much zinc oxide makes for a white, pasty formulation that consumers dislike.

In another formulation, gadusol or gadusporine can be used as a sunscreen at % w/w of 0.5-2.0%, and mixed with other sunscreens, each of which would provide >2 SPF units individually to the topical. In this example, multiple sunscreens are mixed, which reduces the overall volume of each sunscreen in the formulation and reduces or avoids the need for including undesirable sunscreens, such as oxybenzone. In the examples in which gadusol or gadusporine is used as a sunscreen rather than a booster, the gadusol or gadusporine can be in a % w/w of 5-10%, and as a specific example greater than or equal to 3%.

Additional sunscreen formulations are possible. A combination of a gadusol (UVB absorber) and gadusporine(s) (UVA absorber) appears to offer photoprotection over the sun exposure spectrum most associated with skin injury, cancer, and sunburn. A combination of these compounds or derivatives thereof, with or without another nontoxic sunscreen compound (such as a mycosporine or metal oxide), provides sunscreen formulations that may have increased UV blocking or absorbing activity. For example, a formulation of gadusol or gadusporines, or both, in combination with ZnO or with related mycosporines, provides a broad spectrum sunscreen or sunblock. An example formulation (in a cream base) is shown below:

| | |
|---|---|
| Gadusol | 2% |
| Porphyra-334 | 1% |
| Zinc Oxide | 10% |

Porphyra-334 extraction and purification are known and described in, for example, Torres et al., “Porphyra-334, a potential natural source for UVA protective sunscreens,” Photochem. Photobiol. Sci., 2006, 5, 432-435.

The example composition (2% gadusol, 1% porphyra-334, and zinc oxide in a cream base) was applied to VITRO-SKIN® (IMS, Portland ME) for testing. VITRO-SKIN® is an advanced testing substrate that effectively mimics the surface properties of human skin and can be used to assess water resistance of formulations. It has been formulated to have topography, pH, critical surface tension, chemical reactivity and ionic strength that are similar to those of human skin. The resulting SPF was 40.1 with a UVA/UVB ratio of 0.68.

Other example compositions are provided herein. In one example, a composition includes gadusol in combination with one or more of TiO₂, Tinosorb S, Tinosorb M, Ecamsule, oxybenzone, octocrylene, menthyl anthranilate, octinoxate, and avobenzone. Specific examples of composition constituent components and example amounts are provided herein.

It is believed that gadusol and gadusporines also have the capacity to absorb UV radiation and may act as & free radical scavengers. Therefore, gadusol and gadusporines are also antioxidants and may have therapeutic properties akin to other antioxidants, making applications in addition to sunscreens possible, such as in topical treatments, skin care creams, cosmetics and the like.

The cellular toxicity of gadusol was compared to that of a common sunscreen ingredient, oxybenzone. Vero E6 fibroblasts were used to evaluate toxicity. Gadusol was found to be less toxic than oxybenzone. Gadusol was also found not to be mutagenic in the Ames test, nor toxic in the comet assay, nor toxic towards E. coli.

The antioxidant capacity of gadusol and porphyra-334 were evaluated based on the well-known Folin-Ciocalteu method to produce a dose response curve indicating that gadusol at concentrations ranging from 0.3 to 10 mg/ml has the antioxidant power equivalent to a mycosporine compound from “nori” seaweed. When combined, the two compounds have UVA/UVB photo protective activity over a larger spectrum, and the assay showed that the antioxidant power of gadusol is equivalent to that of a mycosporine.

In vivo antioxidant testing revealed that gadusol outperformed porphyra-334. The test is designed to measure antioxidant power but requires also that the anti-oxidant agent cross the cell's lipid boundary layer so that only intracytoplasmic activity is measured. At the highest dose (3.3 mg/ml), both the gadusol and the porphyra-334 caused some lysis of the cells, preventing testing of higher concentrations. However, at about 1 mg/ml, gadusol was a significantly more active antioxidant in vivo than the mycosporine. Where the two were combined, the mycosporine did not interfere with the protective action of the gadusol. Some activity was seen even at concentrations below 0.1 mg/ml.

Addition of an antioxidant could be helpful in increasing the shelf life of gadusol. The stability of gadusol is much higher when stored under acidic conditions. For example, 90% activity was retained after 90 days at pH 2.5. Therefore, storage under acidic conditions may be useful for stabilizing gadusol generally, e.g., prior to introduction into a topical formulation. Combining gadusol with another antioxidant may be useful for extending the shelf life or stability of gadusol in a topical formulation. Storage of gadusol in nitrogen-purged solutions in containers with minimal headspace increased its stability.

The following examples are provided to illustrate sunscreen and topical skin care formulations that may be used. While the examples are provided as sunscreen formulations, it is contemplated that topicals that do not meet regulatory definition(s) of sunscreens may be produced via inclusion of gadusol, MAAs, or a combination thereof, i.e., with or without another approved sunscreen agent. This may include topicals or other formulations that are not marketed as sunscreens but nonetheless would benefit from inclusion of gadusol or MAAs, e.g., antioxidant creams, lotions, etc.

Sunscreen Formulations that Include Gadusol and Zinc Oxide

Gadusol can be used at a low concentration (i.e., up to 0.5% w/w) in conjunction with zinc oxide, Tinosorb M, Tinosorb S, polysilicone-15 in combination with Avobenzone or another UVA filter, MAAs, and gadusporines as an SPF booster because in such formulations, gadusol alone does not contribute significantly enough to SPF activity to be considered an active UV filter, e.g., it contributes <2 SPF units. However, when combined at this low concentration with zinc oxide, Tinosorb M, Tinosorb S, MAAs, gadusporines, and other active sunscreens it increases the total SPF activity of the resulting formulations significantly.

Gadusol can be used at a low concentration (0.5% w/w) with zinc oxide in order to achieve the same SPF activity as formulations containing higher amounts of zinc oxide alone. This approach enables aesthetically pleasing formulations as it decreases the amount of zinc oxide necessary to achieve the target SPF activity.

In one embodiment, an oil-in-water sunscreen formulation contains 0.5% gadusol and 10% zinc oxide (w/w). In vitro SPF testing resulted in a SPF of 26.6±1.8. This same formulation without gadusol resulted in a SPF of 13.9±1.1, thus indicating that the presence of 0.5% gadusol caused a boost of 12.7 SPF units. The example formulation is detailed below.

| Ingredients: | % (w/w) |
|---|---|
| Water (Aqua) | 54.74 |
| Sodium Phytate | 0.05 |
| Xanthan Gum | 0.15 |
| Microcrystalline Cellulose; Cellulose Gum | 1.50 |
| Glycerin | 1.00 |
| Propanediol | 3.00 |
| Ethylhexylglycerin | 1.00 |
| Capylyl Glycol; Ethylhexylglycerin | 1.00 |
| Hydrogenated Lecithin | 0.20 |
| Cetearyl Alcohol; Coco-Glucoside | 4.00 |
| Coco-Glucoside; Coconut Alcohol | 1.50 |
| Cetearyl Alcohol | 1.00 |
| Behenyl Alcohol | 0.25 |
| Zinc Oxide; Caprylic/Capric Triglyceride; | 17.50 |
| Polyhydroxystearic Acid; | |
| lsostearic Acid (of which Zinc Oxide is 10% w/w) | |
| Heptyl Undecylenate | 1.50 |
| Diheptyl Succinate; Caprylol Glycerin/Sebacid Acid | 9.00 |
| Copolymer | |
| Sorbitan Oleate | 0.10 |
| Silica | 1.00 |
| Citric Acid | <0.01 |
| Gadusol | 0.5 |

In another embodiment, gadusol was tested at 0, 0.25, 0.5, 0.75 and 1% with and without 10% zinc oxide in an oil-in-water sunscreen formulation. Both in vivo and in vitro SPF results were comparable as shown in the table below.

| | Formulation | ZnO | Gadusol | In vitro | In vivo |
|---|---|---|---|---|---|
| Base | Code | (%) | (% w/w) | SPF | SPF |
| Cox2 | GLABS16 | 0 | 0 | 1.1 ± 0.0 | 2.93 ± 0.38 |
| Cos2 | GLABS17 | 0 | 0.25 | 3.9 ± 0.4 | 3.6 ± 0.38 |
| Cos2 | GLABS18 | 0 | 0.50 | 3.6 ± 0.6 | 5.11 ± 0.68 |
| Cos2 | GLABS19 | 0 | 0.75 | 2.6 ± 0.2 | 6.30 ± 0.05 |
| Cos2 | GLABS33 | 0 | 1 | NA¹ | 8.74 ± 1.07 |
| Cos1 | GLABS21 | 10 | 0 | 13.9 ± 1.1 | 16.92 ± 1.37 |
| Cos1 | GLABS31 | 10 | 0.25 | NA¹ | 21.11 ± 3.27 |
| Cos1 | GLABS22 | 10 | 0.50 | 26.6 ± 1.8 | 29.7 ± 2.34 |
| Cos1 | GLABS32 | 10 | 0.75 | NA¹ | 35 |
| Cos1 | GLABS34 | 10 | 1 | NA¹ | 51.44 ± 4.43 |

¹The Cos1 sample noted was not tested for in vitro SPF activity.

Cos1 ingredients: 10% ZnO as the only UV filter, plus water, caprylic/capric triglyceride, polyhydroxystearic acid, isostearic acid, diheptyl succinate, capryloyl glycerin/sebacic acid copolymer, cetearyl alcohol, coco-glucoside, propanediol, microcrystalline cellulose, cellulose gum, coconut alcohol, heptyl undecylenate, glycerin, ethylhexylglycerin, caprylyl glycol, and silica with a “5% hole” to allow addition of up to 5% gadusol.

Cos2 ingredients: Water, caprylic/capric triglyceride, diheptyl succinate, capryloyl glycerin/sebacic acid copolymer, cetearyl alcohol, coco-glucoside, propanediol, microcrystalline cellulose, cellulose gum, coconut alcohol, heptyl undecylenate, glycerin, ethylhexylglycerin, caprylyl glycol, silica, behenyl alcohol, and xanthan gum with a “5% hole” to allow the addition of up to 5% gadusol.

In another embodiment, an oil-in-water sunscreen formulation contains 0.5% gadusol, 5% zinc oxide, and a total of 4% Tinosorb M and/or Tinosorb S (w/w), follows:

| Ingredients: | % (w/w) |
|---|---|
| Water (Aqua) | 50.74 |
| Sodium Phytate | 0.05 |
| Xanthan Gum | 0.15 |
| Microcrystalline Cellulose; Cellulose Gum | 1.50 |
| Glycerin | 1.00 |
| Propanediol | 3.00 |
| Ethylhexylglycerin | 1.00 |
| Capylyl Glycol; Ethylhexylglycerin | 1.00 |
| Hydrogenated Lecithin | 0.20 |
| Cetearyl Alcohol; Coco-Glucoside | 4.00 |
| Coco-Glucoside; Coconut Alcohol | 1.50 |
| Cetearyl Alcohol | 1.00 |
| Behenyl Alcohol | 0.25 |
| Zinc Oxide; Caprylic/Capric Triglyceride; | 8.75 |
| Polyhydroxystearic Acid; | |
| lsostearic Acid (of which Zinc Oxide is 5% w/w) | |
| Heptyl Undecylenate | 1.50 |
| Diheptyl Succinate; Caprylol Glycerin/Sebacid Acid | 9.00 |
| Copolymer | |
| Sorbitan Oleate | 0.10 |
| Silica | 1.00 |
| Citric Acid | <0.01 |
| Gadusol | 0.5 |
| Tinosorb M and/or Tinosorb S | 4.0 (total) |

In another embodiment, an oil-in-water sunscreen formulation contains 0.5% gadusol, 5% zinc oxide, and a total of 4% polysilicone-15 (w/w):

| Ingredients: | % (w/w) |
|---|---|
| Water (Aqua) | 50.74 |
| Sodium Phytate | 0.05 |
| Xanthan Gum | 0.15 |
| Microcrystalline Cellulose; Cellulose Gum | 1.50 |
| Glycerin | 1.00 |
| Propanediol | 3.00 |
| Ethylhexylglycerin | 1.00 |
| Capylyl Glycol; Ethylhexylglycerin | 1.00 |
| Hydrogenated Lecithin | 0.20 |
| Cetearyl Alcohol; Coco-Glucoside | 4.00 |
| Coco-Glucoside; Coconut Alcohol | 1.50 |
| Cetearyl Alcohol | 1.00 |
| Behenyl Alcohol | 0.25 |
| Zinc Oxide; Caprylic/Capric Triglyceride; | 8.75 |
| Polyhydroxystearic Acid; | |
| lsostearic Acid (of which Zinc Oxide is 5% w/w) | |
| Heptyl Undecylenate | 1.50 |
| Diheptyl Succinate; Caprylol Glycerin/Sebacid Acid | 9.00 |
| Copolymer | |
| Sorbitan Oleate | 0.10 |
| Silica | 1.00 |
| Citric Acid | <0.01 |
| Gadusol | 0.5 |
| Polysilicone-15 | 4 |

Sunscreen Formulations that Include Gadusol and UVA Filters

Gadusol can be used at >0.5 to 10% as a standalone UVB-protective sunscreen in combination with UVA filters, e.g., avobenzone, zinc oxide, MAAs, and gadusporines to provide full spectrum protection. Specifically, an oil-in-water sunscreen formulation with a cream base contains 0.5-2.0% gadusol and 10% zinc oxide (w/w). Yet another example compound formulation for a topical that includes gadusol is shown below:

| Ingredient | Function | Formula 1 |
|---|---|---|
| petrolatum | base | 3.0 |
| Coconut oil | moisturizer | 3.0 |
| Zinc oxide | UV blocker | 2.0 |
| Gadusol | UV filter | 0.5 to 2.0 |
| Tween-80 | Emulsifier | 1.0 |
| Carbapol-940 | Thickener | 1.0 |
| Triethanolamine | Buffering agent | To pH 7.0 |
| Water | QS to 100 gm | |

Another example embodiment includes the formulation detailed below, composition is given as % (w/w):

| | |
|---|---|
| Titanium dioxide | 5 |
| Zinc oxide | 5 |
| Gadusol | 5-10 |
| Scytonemin | 2 |
| Gadusporine-A* | 0.1 |
| Olive oil | 1 |
| Sorbitan monooleate | 0.5 |
| Glycerol tricaprylate | 1 |
| Caprylic acid | 15 |
| Glycerin | 3 |
| Magnesium sulfate | 0.1 |
| Butylene glycol | 5 |
| Purified water | QS to 100 grams, adjust pH to |
| | 6.2 |

*A mix of MAA or gadusporines could substitute.

Gadusol can be combined with MAAs or gadusporines (e.g., porphyra-334) to create an all-natural, full-spectrum, safe, and effective sunscreen formulation. In the examples below, the porphyra-334 used was 80% pure (i.e., not an extract), although extracts may be utilized if desirable.

In one embodiment, an oil-in-water sunscreen formulation contains 2% gadusol and 1% porphyra-334. In vitro SPF testing resulted in a SPF of 40.1. This same formulation without gadusol or porphyra-334, resulted in an SPF of 16.6, thus indicating that the presence of 1% gadusol and 2% porphyra-334 increased SPF activity by 23.5 units. The example formulation is given below:

| Ingredients: | % (w/w) |
|---|---|
| Water (Aqua) | 52.24 |
| Sodium Phytate | 0.05 |
| Xanthan Gum | 0.15 |
| Microcrystalline Cellulose; cellulose Gum | 1.50 |
| Glycerine | 1.00 |
| Propanediol | 3.00 |
| Ethylhexylglycerin | 1.00 |
| Capylyl Glycol; Ethylhexylglycerin | 1.00 |
| Hydrogenated Lecithin | 0.20 |
| Cetearyl Alcohol; Coco-Glucoside | 4.00 |
| Coco-Glucoside; Coconut Alcohol | 1.50 |
| Cetearyl Alcohol | 1.00 |
| Belenyl Alcohol | 0.25 |
| Zinc Oxide; Caprylic/Capric Triglyceride; | 17.50 |
| Polyhydrostearic Acid; | |
| Isostearic Acid (of which Zinc Oxide is 10 w/w%) | |
| Heptyl Undecylenate | 1.50 |
| Diheptyl Succinate; Caprylol Glycerin/Sebacid Acid | 9.00 |
| Copolymer | |
| Sorbitan Oleate | 0.10 |
| Silica | 1.00 |
| Citric Acid | <0.01 |
| Gadusol | 2 |
| Porphyra-334 | 1 |

Another example embodiment includes the formulation detailed below:

| | |
|---|---|
| Neo-PCl | 5 g (trideceth-9, PEG-5-ethylhexanoate and |
| | water) as hydrating emollient |
| Propylene glycol | 1.25 ml |
| Gadusol | 1.0 ml (as a 10% buffered aqueous solution) |
| Porphyra-334 | 0.75 ml (as a 10% buffered aqueous solution) |

In the U.S., most approved active sunscreens on the FDA sunscreen monograph only protect against UVB. In order to provide a full-spectrum sunscreen, sunscreens typically include either zinc oxide (covers UVA and UVB) or avobenzone. Avobenzone is not photostable which make it less desirable for inclusion.

Gadusporines can be combined with zinc oxide, oxybenzone, octinoxate, octocrylene, titanium dioxide, and/or homosalate to create a full spectrum sunscreen. Gadusporines combined with zinc oxide and/or titanium dioxide provide an “all-natural” sunscreen.

In an embodiment, a formulation includes gadusol in an amount used in the other example formulations described herein, in combination with one or more other actives. By way of example, an embodiment provides a formulation including gadusol and one or more of TiO₂, Tinosorb S, Tinosorb M, Ecamsule, oxybenzone, octocrylene, menthyl anthranilate, octinoxate, and avobenzone. The other actives or components may be provided in an amount suitable to the application, e.g., a conventional amount of sunscreen active may be used for a sunscreen formulation. Moreover, certain ingredients, such as TiO₂, may be omitted or reduced in amount, e.g., where transparency is a characteristic of importance and is more desirable than increased sun protection.

In one embodiment, an oil-in-water sunscreen formulation with a cream base contains 2% gadusol, 10% zinc oxide, and 1% gadusporines. In another embodiment, an oil-in-water sunscreen formulation with a cream base contains 2% gadusol, 10% zinc oxide and 1% mycosporine-like amino acids. Another example embodiment includes the formulations detailed below, composition is given as % (w/w):

| | | For- | For- | For- |
|---|---|---|---|---|
| Ingredient | Function | mula 1 | mula2 | mula 3 |
| Caprylate/capric | Emollient | 1.6 | 1.6 | 1.6 |
| triglycerides | | | | |
| Isopropyl myristate | Permeation | 0 | 5.0 | 0 |
| | enhancer | | | |
| Dimethylisosorbide | Permeation | 0 | 0 | 5.0 |
| | enhancer | | | |
| Beeswax | Lubricant | 1.0 | 1.0 | 1.0 |
| Cetyl palmitate | Emollient/ | 0.5 | 0.5 | 0.5 |
| | stability | | | |
| | enhancer | | | |
| Glutamyl-N-palmitate | Emulsifier | 0.6 | 0.6 | 0.6 |
| (Amisoft ®) | | | | |
| EDTA | Chelating agent | 0.01 | 0.01 | 0.01 |
| Glycerin | Humectant | 3.0 | 3.0 | 3.0 |
| Gadusol | UV Photoprotectant | 0.5-10.0 | 0.5-10.0 | 0.5-10.0 |
| Porphyra-334 or any | UV Photoprotectant | 5.0 | 5.0 | 5.0 |
| MAA or mixture of | | | | |
| MAAor gaduporines | | | | |
| Phenoxyethanol | Preservative | 0.2 | 0.2 | 0.5 |
| Triethanolamine | | To | To | To |
| | | pH 6.5 | pH 6.5 | pH 6.5 |
| Water | QS to 100 gm | | | |

Another example embodiment includes the formulation detailed below, composition is given as % (w/w):

| Ingredient | Function | Formula 1 |
|---|---|---|
| Mineral oil | Emollient | 13.0 |
| Squalane oil | moisturizer | 3.0 |
| Glyceryl monostearate | emulsifier | 1.0 |
| Sorbitan monostearate | Emulsifier/wetting | 1.0 |
| | agent | |
| EDTA | Chelating agent | 0.01 |
| Glycerin | Humectant | 5.0 |
| Gadusol | UV Photoprotectant | 0.5-10.0 |
| Gadusporine A or mix | UV Photoprotectant | 5.0 |
| of | | |
| gadusporines or MAA | | |
| Scytonemin | UV Photoprotectant | 5.0 |
| Phenoxyethanol | Preservative | 0.5 |
| Triethanolamine | | To pH 6.5 |
| Water | QS to 100 gm | |

In an embodiment, composition may be adjusted specifically for sun protection or other indications, e.g., antioxidant activity. For example, an embodiment includes the formulation detailed below, % (w/w):

| | |
|---|---|
| Polysorbates or polyglyceryl oleate emulsifier | 2-10% |
| Potassium L-Glutamyl-N-(1-oxohexadecyl) | 1-4% |
| (CAS#111391-27-6) | |
| L-alanine-N-(1-oxohexadecyl) | 1-4% |
| Emollients 3-10% short chain acetyl triglycerides | 5-10% |
| Grapeseed oil | 5-20% |
| Stearyl palmitate, cetyl alcohol thickener | 0-2% |
| Gadusol | 0.5-10% |
| Gadusporine A* | 1-2% |
| Glycerin, or propylene glycol Humectant | 2-5% |
| Teatree essential oil as a preservative | 0.1-1.0% |
| Water | 50-70% |

*A mix of MAA or gadusporines could substitute

Another example embodiment includes the formulation detailed below (e.g., prepared as a transdermal sunscreen formulation):
Starting with 12 gm of USP (pharmaceutical) water at 30 C, add:
   3 gm Gadusol
   3 gm Gadusporine A*
   0.8 gm Tocopheryl succinate (free acid) as lipophilic ion pair with Gadusporine
   1 gm dodexyl 4,6 diene tyrosine fatty acid amide
   1 gm PEG-300
      * A mix of MAA or gadusporines could substitute

Another embodiment, e.g., for a transdermal formulation, may be prepared as follows:
Starting with 20 gm of USP water at 30 C, add:
   3 gm Gadusol
   3 gm Gadusporine A*
   0.8 gm Tocopheryl succinate (free acid) as lipophilic ion pair with Gadusporine
   1 gm PEG-300
   0.1 gm retinoic acid (active) in 200 ul USP ethanol
   1 gm b-carotene to form an oil-in-water emulsion
   10 mg disodium EDTA
   0.05 gm phenoxyethanol as a preservative
   Adjust pH with TEA/succinic acid to 6.6
      *A mix of MAA or gadusporines could substitute

Gadusol can be used at >0.5 to 10% as a standalone UVB-protective sunscreen. Another example formulation is shown below, % (w/w):

| Polysorbate emulsifier | 2-6% |
|---|---|
| Glyceryl palmitate | 0.1-2% |
| Plant oil emollients | 10-35% |
| Gadusol | 2-10% |
| Short chain triglycerides | 5% |
| Benzyl alcohol-DHA | 0.1-1.0% |
| Fragrance to suit | |
| Water | 60-80% |
| Ingredients: | % (w/w) |
| Water (Aqua) | 62.25 |
| Sodium Phytate | 0.05 |
| Xanthan Gum | 0.15 |
| Microcrystalline Cellulose; Cellulose Gum | 1.50 |
| Glycerin | 1.00 |
| Propanediol | 3.00 |
| Ethylhexylglycerin | 1.00 |
| Capylyl Glycol; Ethylhexylglycerin | 1.00 |
| Hydrogenated Lecithin | 0.20 |
| Cetearyl Alcohol; Coco-Glucoside | 9.00 |
| Coco-Glucoside; Coconut Alcohol | 1.50 |
| Cetearyl Alcohol | 1.00 |
| Behenyl Alcohol | 0.25 |
| Heptyl Undecylenate | 1.50 |
| Diheptyl Succinate; Caprylol Glycerin/Sebacid | 14.00 |
| Acid Copolymer | |
| Sorbitan Oleate | 0.10 |
| Silica | 1.00 |
| Citric Acid | <0.01 |
| Gadusol | 0.5-10% |

Another example formulation is detailed below, % (w/w):

Other Ingredients Added to the Above Formulations to Prevent Degradation or Increase Stability

Similar to addition of gadusol with other actives, addition of other ingredient(s) may be beneficial for other reasons, e.g., to prolong shelf life, increase stability or reduce oxidation rate. For example, antioxidants, singlet oxygen scavengers, or inhibitors of singlet oxygen formation compatible with skincare products may be added to gadusol or any of the formulations described herein, such as: Micah, vitamin A, E, ascorbyl palmitate, MAA, gadusporines, quercetin, crude natural sources or extracts of antioxidants such as seed oils (e.g., sunflower, soybean, other vegetable oils), turmeric, rosemary, ginger, saffron, and fruits or components thereof (e.g., grapes, blackberries, raspberries, strawberries).

Other possibilities to include in the formulations are: Candelilla/Jojoba/Rice Bran, Polyglyceryl-3 Esters, Glyceryl Stearate, Glycerin, Cetearyl Alcohol, Sodium Stearoyl Lactylate, Caprylic/Capric Triglycerides, Butyrospermum Parkii Butter, Vitis vinifera Seed Oil, Prunus armeniaca Kernel Oil, Palmitic/Stearic Triglycerides, Cera alba, Aloe vera extract, Amaranthus caudatus Seed Oil, Hippophae rhamnoides Extract, Argania spinosa Oil, Helianthus annuus Seed Oil, Lycium barbarum Fruit Extract, Mimosa tenuiflora Bark Extract, Sodium Benzoate, Potassium Sorbate, Mixed tocopherols, Cocos nucifera Oil, Prunus Amygdalus dulcis Oil, Musa sapientum Fruit Extract, Olea europaea Leaf Extract, Sambucus nigra Flower Extract, Camelia sinensis Leaf Extract, Glycyrrhiza gia bra Extract, Cyamopsis tetragonoloba Gum, Glycyrrhetinic Acid, Phytic Acid, Citrus aurantifolia Peel Oil, Helianthus annuus Seed Oil, resveratrol, stilbenes generally, flavonoidsas a class, and Rosmarinus officinalis Leaf Extract, for example.

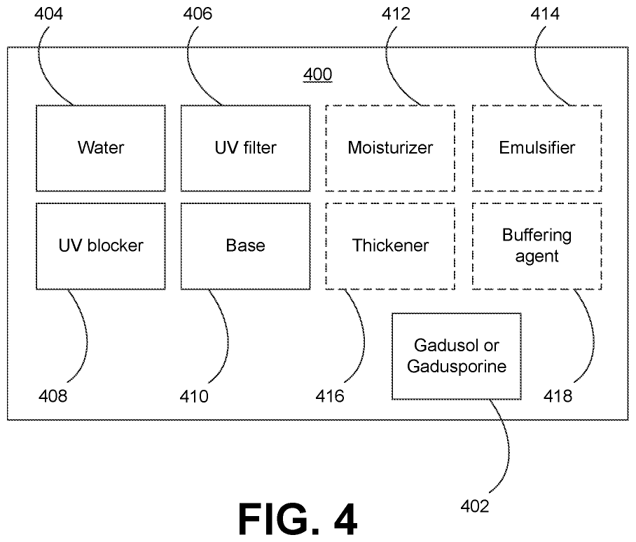

FIG. 4 shows an example compound formulation 400, as disclosed herein. Specific example formulations are discussed above. The example formulation 400 includes gadusol or gadusporine 402 and in some examples includes both gadusol and gadusporine 402. In those examples, one or the other of the gadusol and the gadusporine can be a booster—have a ≤0.5% w/w, for example—while the other can be a sunscreen—have a >0.5% w/w or >2.0 SPF units as a standalone ingredient. In other examples, gadusol and gadusporine are both included in the formulation and are either both included at 0.5-2.0% w/w or >0.5% w/w. The compound formulation 400 also includes water 404, a UV filter 406, a UV blocker 408, a base 410, a moisturizer 412, an emulsifier 414, a thickener 416, and a buffering agent 418. Examples of each is described above.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing the invention in diverse forms thereof.

1-20. (canceled)

21. A zinc oxide-containing sunscreen composition generated by the steps of:
a. preparing a first formulation having zinc oxide as a sunscreen active ingredient, wherein the composition comprises:
i. a cosmetic base comprising an oil in water emulsion; and
ii. a mineral sunscreen active ingredient comprising zinc oxide;
iii. a first sun protection factor (SPF) value; and
b. preparing a second formulation having zinc oxide as the sunscreen active ingredient, wherein the composition comprises:
1. the cosmetic base comprising the oil in water emulsion;
2. the mineral sunscreen active ingredient comprising zinc oxide;
3. an additive having an absorption in the UVB range; and
4. a second SPF value;
c. determining the first and second SPF values for each of the first and second formulations;
d. comparing the first and second SPF values;
e. determining an effectiveness of the additive as a sunscreen booster material;
f. generating an amount of the additive suitable to provide a sunscreen boosting effect to a sunscreen composition comprising the cosmetic base and an amount of zinc oxide as a sunscreen active material and
g. preparing a third formulation comprising the first formulation and the additive amount from step (f).

22. The method of claim 21, wherein the additive is gadusol.

23. The method of claim 21, further comprising preparing a plurality of the second formulations, each including different amounts of the additive, wherein each of the plurality of second formulations has a SPF value associated with each different amount of the additive incorporated in each of the formulations.

24. The method of claim 21, wherein the additive has a UV absorbance maximum of 296 nm.

25. The method of claim 21, wherein the additive is not porphyra-334 or shinorine.

26. The method of claim 21, wherein the additive is not derived from the red algae Porphyra umbilicalis.

27. The method of claim 21, wherein the effectiveness of the additive as a sunscreen booster material comprises determining that the second SPF value is greater than the first SPF.

28. The method of claim 26, wherein the amount of SPF value increase is less than 2 SPF units, thereby generating a determination that the additive is a sunscreen booster material when incorporated in an oil in water formulation comprising zinc oxide.

29. The method of claim 21, further comprising:
a. preparing a third formulation comprising:
i. the cosmetic base comprising the oil in water emulsion;
ii. an amount of the additive, wherein, the third formulation does not comprise the zinc oxide; and
b. generating a third SPF value for the third formulation.

30. The method of claim 28, further comprising determining that the wherein the amount of SPF value increase is less than 2 SPF units, thereby generating a determination that the additive is a sunscreen booster material when incorporated in an oil in water formulation comprising zinc oxide.

31. The method of claim 21, wherein the additive is gadusporine.

32. A sunscreen formulation prepared by the method of claim 21.

33. The sunscreen formulation of claim 29, wherein the additive is gadusol.

34. The sunscreen formulation of claim 33, wherein the gadusol is added at from 0.25% to 1% by weight of the sunscreen formulation.

35. The sunscreen formulation of claim 33 comprising a broad spectrum sunscreen having absorbance in both the UVA and UVB region.
